(19) United States
(12) Patent Application Publication
(10) Pub. No.: US 2025/0312566 A1
(43) Pub. Date: Oct. 9, 2025

(54) Catheter Placement System and Method

(71) Applicant: C. R. Bard, Inc., Franklin Lakes, NJ (US)

(72) Inventor: Mark S. Kelemen, Woods Cross, UT (US)

(21) Appl. No.: 18/866,898

(22) PCT Filed: May 27, 2022
(86) PCT No.: PCT/US22/31434

§ 371 Date: Nov. 18, 2024

(51) Int. Cl. A61M 25/01; A61M 25/00

(52) U.S. Cl. CPC A61M 25/0102; A61M 25/003; A61M 2025/0037

(57) ABSTRACT

Embodiments disclosed herein are directed to a catheter placement system including a dual lumen catheter, or similar device, and a catheter stiffening system configured to facilitate placement of the catheter. The catheter stiffening system can include a first stylet disposed in a first catheter lumen and a second stylet disposed in a second catheter lumen. A guidewire can extend through a stylet lumen of the second stylet and a channel of the first stylet. A distal portion of the first stylet can include a skived portion and a distal portion of the second stylet can include a notched portion. As such, the distal portions of the first stylet and the second stylet can interlock and align the stylet lumen with the channel. The guidewire can extend through both the stylet lumen and the channel to further secure the distal portions of the stylets together.

### SUMMARY

Briefly summarized, embodiments of the present invention are directed to a catheter placement system and associated methods thereof. The system can include a split-tip hemodialysis catheter, or similar vascular access device, and a catheter stiffening system configured to facilitate placement of the split-tip catheter. The catheter stiffening system can include a first stylet disposed in a first catheter lumen and a second stylet disposed in a second catheter lumen. A guidewire can extend through one or both of the stylets and can extend to a target location within the vasculature. A distal portion of the first stylet can include a skived portion and a distal portion of the second stylet can include a notched portion. As such, the distal portions of the first stylet and the second stylet can interlock and align a lumen of the second stylet with a channel of the first stylet. The guidewire can extend through both the stylet lumen and the channel to further secure the distal portions of the stylets together.

Advantageously, embodiments disclosed herein allow for placement of catheters that include intricate tip designs, and allows these catheters to be placed over a single guidewire. For example, embodiments disclosed herein allow for placement of non-rigid, flexible tips, split tips, or the like, as the stiffening system provides increased rigidity and alignment with the lumen of the vasculature. Further, embodiments allow for placement of radially protruding preset tip geometries, e.g. Y-shaped, or U-shaped split tip-catheters, by elastically deforming the split-tips to a smaller cross-sectional profile during placement, e.g. tapered. Once placed within the vasculature, the stiffening system can be removed and the split-tip can return to the Y-shaped structure.

Advantageously, any axial movement applied to one of the first stylet or the second stylet translates the movement directly to the other stylet, preventing kinking or severing of the guidewire. Further, one of the first stylet or the second stylet can be urged distally or proximally relative to the other stylet to facilitate steering a distal portion of the catheter assembly through tortuous vascular pathways without applying pressure to the guidewire. This can allow the assembly to slide over the guidewire, as well as mitigating kinking or severing of the guidewire.

Disclosed herein is a catheter placement system including, a catheter defining a first catheter lumen and a second catheter lumen, a first stylet disposed within the first catheter lumen and including a skived portion defining a proximally facing abutment surface, and a channel extending from the abutment surface to a distal tip surface, a second stylet disposed within the second catheter lumen and defining a stylet lumen, the second stylet including a notch having a distally facing notch surface and configured to abut against the proximally facing abutment surface to align the stylet lumen with the channel, and a guidewire extending through the stylet lumen and through the channel to extend distally of the distal tip surface of the first stylet.

In some embodiments, the notch extends from a side surface of the second stylet radially inward to define the distally facing notch surface.

In some embodiments, the notch surface extends parallel to a distal tip surface of the second stylet and is disposed proximally of the distal tip surface.

In some embodiments, one or both of the notch surface and the abutment surface are angled relative to a lateral axis of the second stylet or the first stylet respectively to form a “French cleat” engagement.

In some embodiments, the stylet lumen communicates with one or both of the notch surface and the distal tip surface of the second stylet.

In some embodiments, an inner diameter of one or both of the stylet lumen and the channel is equal to or slightly larger than an outer diameter of the guidewire.

In some embodiments, one or both of the skived portion and the notch include one or more protrusions, detents, interlocking teeth, “lock-and-key” structures, clip, barbed, or latch structures, configured to releasably engage the skived portion with the notch.

In some embodiments, the notch is configured to engage the skived portion to define a tapered outer profile to a distal portion of an assembly of the first stylet and the second stylet.

Also disclosed is a method of placing a catheter within a vasculature including, advancing a catheter assembly over the guidewire, the catheter assembly having a catheter defining a first catheter lumen and a second catheter lumen, a first stylet disposed within the first catheter lumen and including a skived portion defining a proximally facing abutment surface, and a channel extending from the abutment surface to a distal tip surface, and a second stylet disposed within the second catheter lumen and defining a stylet lumen, the second stylet including a notch having a distally facing notch surface, urging one or both of the first stylet and the second stylet distally, abutting the notch surface against the abutment surface, and sliding one or both of the first stylet and the second stylet over the guidewire.

In some embodiments, the notch surface engages the abutment surface to align the stylet lumen with the channel and slidably engage the guidewire.

In some embodiments, one or both of the notch surface and the abutment surface are angled relative to a lateral axis to form a “French cleat” wherein distal movement of the second stylet or proximal movement of the first stylet further engages the notch surface with the abutment surface.

In some embodiments, urging the second stylet distally relative to the first stylet causes the notch surface to impinge on the abutment surface and urge a distal portion of the first stylet and a second stylet assembly in a first direction.

In some embodiments, urging the first stylet distally relative to the second stylet urges a distal portion of the first stylet and a second stylet assembly in a second direction, opposite the first direction.

In some embodiments, urging the first stylet proximally relative to the second stylet causes the notch surface to impinge on the abutment surface and urges the distal portion of the first stylet and a second stylet assembly in the first direction.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a schematic diagram illustrating a partially cutaway side view of a catheter apparatus of a catheter placement system, in accordance with embodiments of the present disclosure.

FIG. 2A shows a lateral side view of a distal portion of a catheter placement system, in accordance with embodiments of the present disclosure.

FIG. 2B shows a transverse side view of a distal portion of a catheter placement system, in accordance with embodiments of the present disclosure.

FIGS. 3A-3B show perspective views of a distal portion of a first stylet of a catheter placement system, in accordance with embodiments of the present disclosure.

FIG. 4 shows a perspective view of a distal portion of a second stylet of a catheter placement system, in accordance with embodiments of the present disclosure.

FIG. 5 shows a schematic side view of a distal portion of a first stylet and a distal portion of a second stylet, in accordance with embodiments of the present disclosure.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention, and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

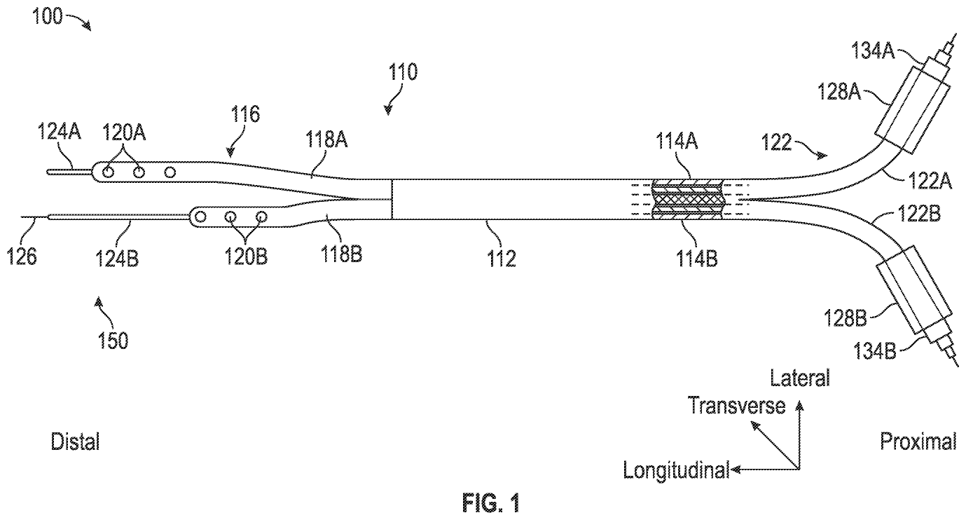

To assist in the description of embodiments described herein, as shown in FIG. 1, a longitudinal axis extends substantially parallel to an axial length of the catheter. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

Referring to FIG. 1, this schematic diagram illustrates, in a partially cutaway side view, a catheter placement system (“system”) 100 such as a hemodialysis catheter insertion system, in accordance with an embodiment of the present disclosure. The catheter placement system 100 can generally include a catheter 110 and a stiffening system 150. As will be appreciated the catheter 110 is exemplary and embodiments disclosed herein can be used with various vascular access devices or catheters, such as dual or multi-lumen catheters, central vascular catheter (CVC), peripherally inserted central catheters (PICC), midline catheters, or the like.

In an embodiment, the catheter 110 can include a catheter body 112 defining two internal lumens 114A and 114B. The catheter body 112 can include a “split-tip” distal end 116 in which the body (and lumens) separate into two distal tip portions, 118A and 118B, which form a single-lumen distal blood return extension tube and a single-lumen distal blood removal extension tube, respectively. The split-tips can, but need not have, one or more side ports 120A and 120B, in fluid communication with one or the other of the lumens 114A and 114B to facilitate respective blood return and removal during hemodialysis.

Still referring to FIG. 1, alternatively, or in conjunction with side ports 120, the distal ends 116 can be open to provide fluid passageways for blood removal and return. The proximal end 122 of the catheter body 112 can also be split into separate segments 122A and 122B, e.g. extension legs, and terminate with two couplers 128A and 128B, which can include couplings 134A and 134B, such as luer-locks or the like, to couple the catheter system 100 to a hemodialysis machine (not shown) in which blood is circulated and purified. Proximal segments 122A, 122B, thus, respectively provide a single-lumen proximal blood return extension tube and a single-lumen proximal blood removal extension tube. However, an additional lumen or lumens are also possible.

Still referring to FIG. 1, the overall system 100 of the present disclosure can also include a catheter stiffener system 150 which can include one or more stylets 124 and a guidewire 126. In an embodiment, a first stylet 124A can be disposed within a first catheter lumen 114A and a second stylet 124B can be disposed in a second catheter lumen 114B. In an embodiment, the catheter body 112 can be formed of a relatively flexible material such as a plastic, polymer, elastomer, rubber, silicone, silicone rubber, polyurethane, or other biocompatible composition, e.g., having a stiffness in the range of about 65 to about 85 durometers. Preferably, the stylet(s) 124 comprise a relatively stiffer material such as a plastic, polymer, polyethylene, metal, alloy, composite, or other bio-compatible material. Advantageously, the stiffening assembly 150 can facilitate preventing kinking of the catheter body 112 during insertion.

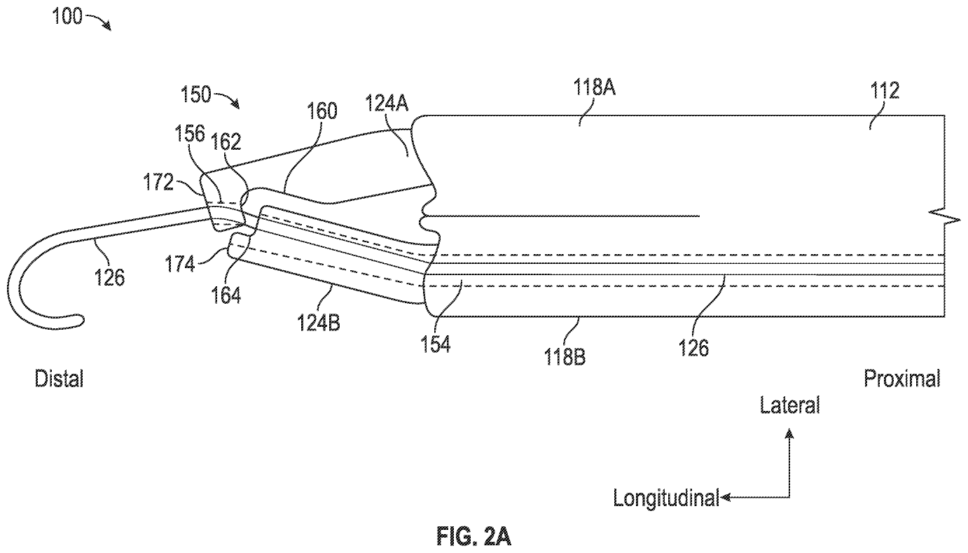

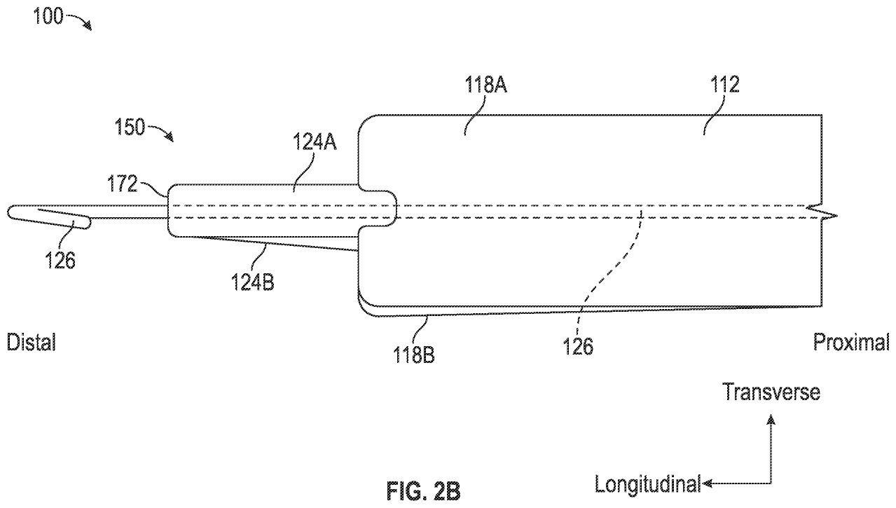

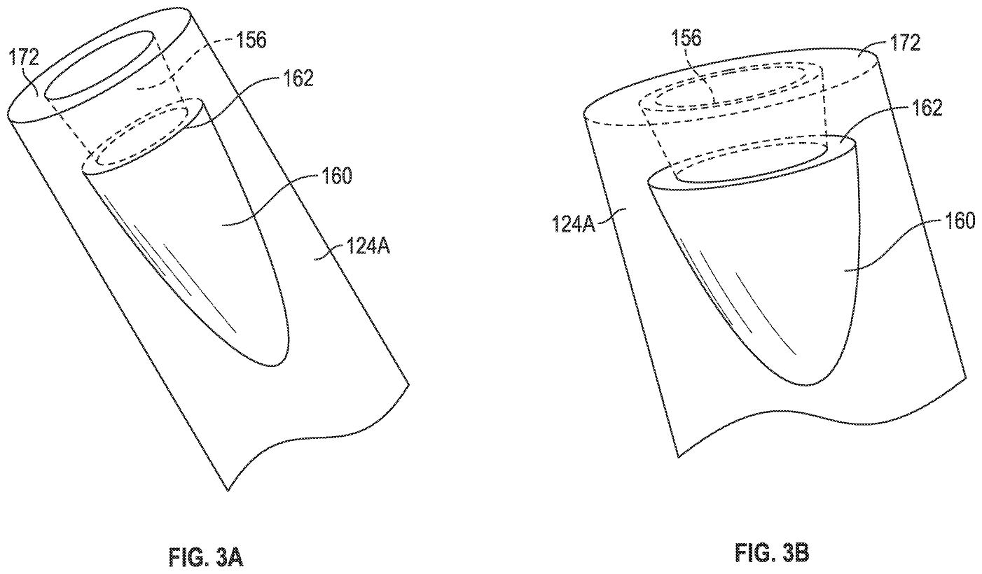

FIGS. 2A-4 show further details of a distal portion of the catheter placement system 100. FIG. 2A shows a lateral side view of a distal portion of the catheter placement system 100 and FIG. 2B shows a transverse side view of a distal portion of the catheter placement system 100. In an embodiment, the second stylet 124B can define a stylet lumen 154 extending axially, and extending to a distal tip 174 of the second stylet 124B. In an embodiment, the stylet lumen 154 can extend to a proximal end of the second stylet 124B.

In an embodiment, a distal portion of the first stylet 124A can include a channel 156 extending therethrough. The channel 156 can extend from a distal tip 172 of the first stylet 124A to a point that is proximal of the distal tip 172. The guidewire 126 can extend through the stylet lumen 154 and extend from a distal tip 174 of the second stylet 124B. The guidewire 126 can then extend through the channel 156 of the first stylet 124A and can extend distally of the distal tip 172 of the first stylet 124A to a target location within the vasculature.

In an embodiment, the first stylet 124A can include a skived portion 160 disposed proximally of the channel 156. A surface of the skived portion 160 can extend at a shallow angle relative to a longitudinal axis of the first stylet 124A and can extend radially inward from an outer-most side surface of the first stylet 124A. A distal end of the skived portion 160 can extend at an angle, e.g. perpendicular, to the longitudinal axis of the first stylet 124A to define a substantially proximally facing abutment surface 162. The channel 156 can extend from the abutment surface 162 to the distal tip surface 172 and can define an inner diameter that is equal to or slightly larger than an outer diameter of the guidewire 126. As such, the guidewire 126 can slidably engage the channel 156.

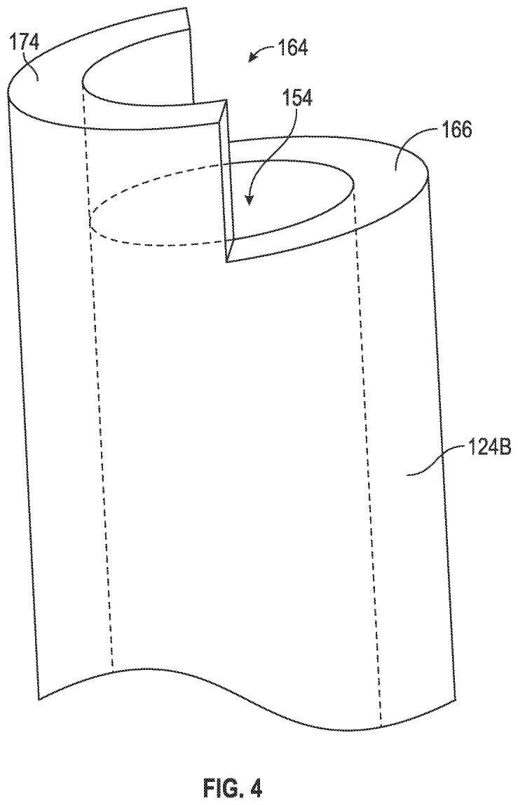

In an embodiment, as shown in FIGS. 2A and 4, a distal tip surface 174 of the second stylet 124B can include a notch 164 extending at an angle, e.g. perpendicular, to a longitudinal axis and extending radially inward from an outer-most side surface of the second stylet 124B. The notch 164 can extend inward from an inner side of the second stylet 124B, i.e. from a side of the second stylet 124B that is adjacent the first stylet 124A. As such, a distally facing notch surface 166 can be disposed proximally of a distally facing distal tip surface 174 of the second stylet 124B. The stylet lumen 154 can communicate with one or both of the distal tip surface 174 and the distally facing notch surface 166.

In an embodiment, a portion of the notch 164 can engage the skived portion 160 such that the distally facing notch surface 166 abuts against the proximally facing abutment surface 162 and the stylet lumen 154 aligns with the channel 156 of the first stylet 124A. In an embodiment, one or both of the notch 164 and the skived portion 160 can include one or more engagement structures configured to secure the first stylet 124A and the second stylet 124B in an interference fit or snap fit engagement. In an embodiment, one or both of the notch surface 166 and the abutment surface 162 can include one or more protrusions, detents, interlocking teeth, “lock-and-key” structures, clip, barbed, or latch structures, or similar structures to facilitate a securing engagement between the notch surface 166 and the abutment surface 162 and to align the lumen 154 with the channel 156.

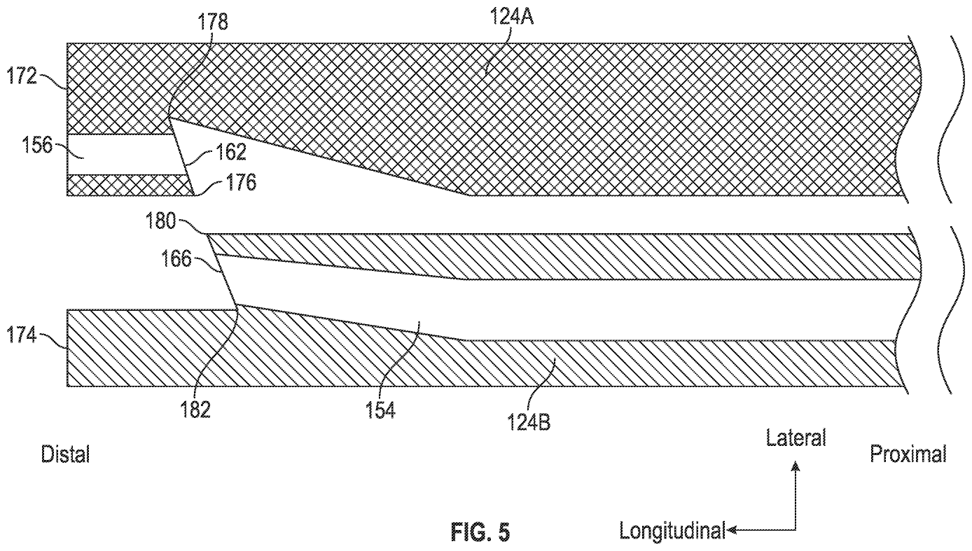

In an embodiment, as shown in FIG. 5, the abutment surface 162 can be angled relative to the lateral axis such that a radially outer-most edge 176 of the abutment surface 162 is disposed proximally of a radially inner-most edge 178. Similarly, the notch surface 166 can be angled relative to the lateral axis such that a radially outer-most edge 180 of the notch surface 166 is disposed distally of a radially inner-most edge 182. As such, the notch surface 166 can engage the abutment surface 162 in a “French cleat” engagement where any distal movement of the second stylet 124B or any proximal movement of the first stylet 124A can further secure the engagement therewith, ensuring the stylet lumen 154 remains aligned with the channel 156.

With continued reference to FIG. 2A, in an embodiment an inner diameter of one or both of the channel 156 and the stylet lumen 154 can be equal to or slightly larger than an outer diameter of the guidewire 126. As such, with the guidewire 126 extending through both the stylet lumen 154 and the channel 156, the guidewire 126 can facilitate aligning the notch surface 166 with the abutment surface 162. Advantageously, a distal portion of the second stylet 124B can interlock with a distal portion of the first stylet 124A, as described herein, and the guidewire 126 can extend through both distal portions, securing the respective distal portions together.

In an embodiment, the notch 164 and skived portion 160 can engage to allow a portion of the second stylet 124B to directly engage a portion of the first stylet 124A. As such, any axial movement of the first stylet 124A applies pressure directly to a surface of the second stylet 124B. Similarly, any axial movement of the second stylet 124B applies pressure directly to a surface of the first stylet 124A. Advantageously, the interaction between the notch 164 and skived portion 160 mitigates applying pressure, e.g. sheering pressure, etc., to the guidewire 126. This can prevent kinking of the guidewire 126 that can inhibit removal of the guidewire 126 through one or both of the channel 156 and stylet lumen 154, and prevent releasing the stylets 124A, 124B for subsequent removal. Further, this can prevent sheering or rupture of a distal portion of the guidewire 126 within the vasculature, causing various complications for the patient.

Advantageously, the interaction between the notch 164 and skived portion 160 can further allow the stylets 124A, 124B to push against each other to help steer a distal portion of the system 100. For example, advancing the second stylet 124B distally relative to the first stylet 124A can cause the notch surface 166 to abut against the abutment surface 162 and urge the distal portion of the catheter 110 in a first direction. Similarly, advancing the first stylet 124A distally relative to the second stylet 124B can cause a surface of the skived portion 160 to engage a surface of the second stylet 124B and urge the distal portion of the catheter 110 in a second direction, opposite the first direction. Rotating the system 100 about the longitudinal axis can facilitate steering the distal portion of the catheter 110 in additional directions.

Advantageously, the stylets 124A, 124B can steer the catheter 110 while mitigating any pressure applied to the guidewire 126 which may cause friction between the stylets 124A, 124B and the guidewire 126 and prevent the system 100 from sliding over the guidewire 126. Further, the stylets 124A, 124B can steer the catheter 110 while mitigating kinking or rupture of the guidewire 126, as described herein. Further, the stylets 124A, 124B can facilitate steering the catheter 110 through tortuous vascular pathways over the guidewire 126.

Advantageously, the stiffening system 150 allows for placement of a catheter 110 having intricate tip designs, and allows these catheters 110 to be placed over a single guidewire. For example, the stiffening system 150 allows for placement of non-rigid, flexible tips, split tips, or the like, as the stiffening system 150 provides increased rigidity and alignment with the lumen of the vasculature. Further, embodiments allow for placement of radially protruding preset tip geometries, e.g. catheter tips that define a Y-shaped, or U-shaped, split tip design when at rest. The stiffening system 150 elastically deforms the split-tips to a smaller cross-sectional profile to facilitate negotiating tortuous vascular pathways. Once in place, the stiffening system 150 can be removed, as described herein, and the split-tip can return to the Y-shaped/U-shaped structure.

In an exemplary method of use, a catheter placement system 100 is provided, as described herein. The system 100 can include a catheter 110 having a first stylet 124A disposed in a first lumen 114A and a second stylet 124B disposed in a second lumen 114B. A guidewire 126 can extend through a stylet lumen 154 of the second stylet 124B and through a channel 156 of the first stylet 124A and extend distally to a target location within the vasculature. The catheter 110 and stylet 124 assembly can then be advanced over guidewire 126 and into the vasculature. Advantageously, the notched portion 164 of the second stylet 124B can be received within a skived portion 160 of the first stylet 124A to define a tapered outer profile to a distal portion of the stylet 124A, 124B and guidewire 126 assembly and facilitate advancement into and through the vasculature. Further, the notch surface 166 can abut against the abutment surface 162 and mitigate applying pressure to the guidewire 126 that may kink or rupture the guidewire 126.

The stylets 124A, 124B can provide stiffening to the catheter 110 to allow axial pressure to be applied to the catheter 110 without kinking or collapsing of the catheter 110. The axial pressure can advance the catheter 110 over the guidewire to a target location within the vasculature. Advantageously, applying pressure to one or other of the first stylet 124A or the second stylet 124B can facilitate steering a distal portion of the catheter 110 to negotiate tortuous vascular pathways. Further, the interaction between the notch 164 and skived portion 160 can allow the first stylet 124A and the second stylet 124B to steer the catheter 110 while mitigating applying pressure to the guidewire 126, as described herein. Advantageously, the interlocking stylets 124A, 124B and guidewire 126 assembly can maintain the split tips 118A, 118B of the catheter 110 adjacent to each other during placement to facilitate advancing through the vasculature.

Once the catheter 110 has been placed at the target location, the guidewire 126 can be withdrawn proximally. A distal tip of the guidewire 126 can be withdrawn through the channel 156, through the stylet lumen 154 and removed proximally from the catheter 110. With the guidewire 126 removed, the distal portion of the second stylet 124B can disengage the distal portion of the first stylet 124A. Each of the first stylet 124A and the second stylet 124B can be withdrawn proximally through the respective first lumen 114A and second lumen 114B leaving the catheter 110 in place within the vasculature.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A catheter placement system, comprising:
a catheter defining a first catheter lumen and a second catheter lumen;
a first stylet disposed within the first catheter lumen and including a skived portion defining a proximally facing abutment surface, and a channel extending from the abutment surface to a distal tip surface;
a second stylet disposed within the second catheter lumen and defining a stylet lumen, the second stylet including a notch having a distally facing notch surface and configured to abut against the proximally facing abutment surface to align the stylet lumen with the channel; and
a guidewire extending through the stylet lumen and through the channel to extend distally of the distal tip surface of the first stylet.

2. The catheter placement system according to claim 1, wherein the notch extends from a side surface of the second stylet radially inward to define the distally facing notch surface.

3. The catheter placement system according to claim 1, wherein the notch surface extends parallel to a distal tip surface of the second stylet and is disposed proximally of the distal tip surface.

4. The catheter placement system according to claim 1, wherein one or both of the notch surface and the abutment surface are angled relative to a lateral axis of the second stylet or the first stylet respectively to form a “French cleat” engagement.

5. The catheter placement system according to claim 1, wherein the stylet lumen communicates with one or both of the notch surface and the distal tip surface of the second stylet.

6. The catheter placement system according to claim 1, wherein an inner diameter of one or both of the stylet lumen and the channel is equal to or slightly larger than an outer diameter of the guidewire.

7. The catheter placement system according to claim 1, wherein one or both of the skived portion and the notch include one or more protrusions, detents, interlocking teeth, “lock-and-key” structures, clip, barbed, or latch structures, configured to releasably engage the skived portion with the notch.

8. The catheter placement system according to claim 1, wherein the notch is configured to engage the skived portion to define a tapered outer profile to a distal portion of an assembly of the first stylet and the second stylet.

9. A method of placing a catheter within a vasculature, comprising:
advancing a catheter assembly over the guidewire, the catheter assembly comprising:
a catheter defining a first catheter lumen and a second catheter lumen;
a first stylet disposed within the first catheter lumen and including a skived portion defining a proximally facing abutment surface, and a channel extending from the abutment surface to a distal tip surface; and
a second stylet disposed within the second catheter lumen and defining a stylet lumen, the second stylet including a notch having a distally facing notch surface;
urging one or both of the first stylet and the second stylet distally;
abutting the notch surface against the abutment surface; and
sliding one or both of the first stylet and the second stylet over the guidewire.

10. The method according to claim 9, wherein the notch surface engages the abutment surface to align the stylet lumen with the channel and slidably engage the guidewire.

11. The method according to claim 9, wherein one or both of the notch surface and the abutment surface are angled relative to a lateral axis to form a “French cleat” wherein distal movement of the second stylet or proximal movement of the first stylet further engages the notch surface with the abutment surface.

12. The method according to claim 9, wherein urging the second stylet distally relative to the first stylet causes the notch surface to impinge on the abutment surface and urge a distal portion of the first stylet and a second stylet assembly in a first direction.

13. The method according to claim 12, wherein urging the first stylet distally relative to the second stylet urges a distal portion of the first stylet and a second stylet assembly in a second direction, opposite the first direction.

14. The method according to claim 12, wherein urging the first stylet proximally relative to the second stylet causes the notch surface to impinge on the abutment surface and urges the distal portion of the first stylet and a second stylet assembly in the first direction.
